(19) United States
(12) Patent Application Publication
(10) Pub. No.: US 2011/0038829 A1
(43) Pub. Date: Feb. 17, 2011

(54) METHODS AND MATERIALS FOR TREATING DIARRHEA

(75) Inventor: Michael L. Camilleri, Rochester, MN (US)

Correspondence Address: FISH & RICHARDSON P.C. (TC), PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 12/851,216

(22) Filed: Aug. 5, 2010

Related U.S. Application Data
(60) Provisional application No. 61/233,409, filed on Aug. 12, 2009.

(51) Int. Cl. A61K 31/785; A61P 1/12

(52) U.S. Cl. 424/78.38

(57) ABSTRACT

This document relates to methods and materials for administering bile acid sequestrants to treat conditions associated with diarrhea. For example, formulations designed for the delivery of a bile acid sequestrant (e.g., colesevelam) to treat diarrhea are provided.

### CROSS-REFERENCE TO RELATED APPLICATIONS

This application claims the benefit of priority to U.S. Provisional Application Ser. No. 61/233,409, filed Aug. 12, 2009. The disclosure of the prior applications is considered part of (and is incorporated by reference in) the disclosure of this application.

### STATEMENT AS TO FEDERALLY SPONSORED RESEARCH

This invention was made with government support under DK054681 awarded by National Institute of Diabetes and Digestive and Kidney Diseases. The government has certain rights in the invention.

### BACKGROUND

1. Technical Field

This document relates to methods and materials for administering a bile acid sequestrant (e.g., colesevelam) to treat conditions associated with diarrhea (e.g., bile acid malabsorption induced diarrhea). For example, this document provides methods for treating bile acid malabsorption induced diarrhea using colesevelam.

2. Background Information

Bile acids are formed in the liver from cholesterol and have a variety of physiologic functions from cholesterol elimination to enhancement of lipid absorption in the small intestine. Up to 95% of bile acids secreted into bile are actively reabsorbed in the terminal ileum.

### SUMMARY

This document relates to methods and materials for administering a bile acid sequestrant (e.g., colesevelam) to treat conditions associated with diarrhea (e.g., bile acid malabsorption induced diarrhea).

In general, one aspect of this document features a method for treating a diarrhea condition. The method comprises, or consists essentially of, administering to a mammal having the diarrhea condition a composition comprising a bile acid sequestrant. The mammal can be a human. The diarrhea condition can be bile acid malabsorption induced diarrhea. The bile acid sequestrant can be colesevelam. The composition can be administered to the mammal under conditions wherein the amount of the bile acid sequestrant in the composition is between about 1 gram and about 5 grams. The composition can be administered to the mammal between once every other day to four times a day. The method can comprise assessing the mammal before the administering step to determine if the mammal comprises a serum level of 7αC4 greater than about 25 ng/mL of serum, wherein the presence of the serum level indicates that the mammal is to be administered the bile acid sequestrant. The serum level of 7αC4 can be a fasting serum level of 7αC4. The method can comprise assessing the mammal before the administering step to determine if the mammal comprises a serum level of 7αC4 greater than about 30 ng/mL of serum, wherein the presence of the serum level indicates that the mammal is to be administered the bile acid sequestrant.

In another aspect, this document features a method for treating a diarrhea condition. The method can comprise, or consist essentially of, (a) identifying a mammal having the diarrhea condition and a serum level of 7αC4 greater than about 25 ng/mL of serum, and (b) administering a composition comprising a bile acid sequestrant to the identified mammal under conditions wherein the severity of the diarrhea condition is reduced. The serum level of 7αC4 can be a fasting serum level of 7αC4.

In another aspect, this document features a method for treating a diarrhea condition. The method comprises, or consist essentially of, (a) identifying a mammal having (i) the diarrhea condition, (ii) a serum level of 7αC4 greater than about 20 ng/mL of serum, and (iii) a potential bile acid malabsorption condition based on a fecal bile acid excretion test or ⁷⁵SeHCAT retention test, and (b) administering a composition comprising a bile acid sequestrant to the identified mammal under conditions wherein the severity of the diarrhea condition is reduced. The fecal bile acid excretion test can be a 48 hour fecal bile acid excretion test. The ⁷⁵SeHCAT retention test can be a ⁷⁵SeHCAT retention test at 7 days.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### DESCRIPTION OF DRAWINGS

FIG. 1. Study flow chart and demographics of participants in CDC study.

FIG. 2. Study flow chart and demographics of participants in colesevelam study.

FIG. 3. Effects of NaCDC on colonic transit (GC) at 24 and 48 hours, and stool form and frequency. Data show least square means±SEM. There is an overall acceleration of colonic transit, and this is significantly greater with the 1000 mg dose of Na CDC (p<0.0001 vs. placebo) relative to the 500 mg dose (p<0.01 vs. placebo), particularly evident with the transit data at 24 hours (overall p<0.05, 1000 mg vs. 500 mg). The acceleration of colonic transit was associated with an increase in stool frequency (p<0.001) and a more liquid consistency stool (p<0.0001).

FIG. 4. Effects of placebo or colesevelam on overall colonic transit at 24 hours in individual patients. 7/12 participants in the colesevelam arm had reduced colonic transit by >0.7.

FIG. 5. Effects of colesevelam and placebo on ascending colon emptying t_{1/2} in relation to the rank of baseline fasting serum 7αC4. Note that the ascending colon emptying times are longest on colesevelam treatment in the patients with the higher rank levels of fasting serum 7αC4.

### DETAILED DESCRIPTION

This document relates to methods and materials for administering a bile acid sequestrant (e.g., colesevelam) to treat conditions associated with diarrhea (e.g., bile acid malabsorption induced diarrhea). For example, this document provides methods for treating diarrhea in a mammal, including without limitation, a human, dog, cat, horse, pig, monkey, or sheep. Examples of diarrhea conditions that can be treated as described herein include, without limitation, bile acid malabsorption induced diarrhea, ileal resection diarrhea, radiation ileitis, Crohn's ileitis, acute Yersinia ileitis, diabetic diarrhea, diarrhea associated with small bowel bacterial overgrowth, irritable bowel syndrome with diarrhea, diarrhea-predominant irritable bowel syndrome, functional diarrhea, and pancreatic transplant associated diarrhea. Examples of bile acid sequestrants that can be used as described herein include, without limitation, colesevelam, cholestyramine, colestipol, and chitosan. In some cases, the methods provided herein can include identifying a mammal (e.g., human) to be treated. For example, a method provided herein can include assessing the level of serum 7αC4 (e.g., fasting serum 7αC4) within a mammal. In some cases, a level greater than about 20 ng of 7αC4 per mL of serum (e.g., greater than about 25 ng/mL, greater than about 26 ng/mL, greater than about 27 ng/mL, greater than about 28 ng/mL, greater than about 29 ng/mL, greater than about 30 ng/mL, greater than about 31 ng/mL, greater than about 32 ng/mL, greater than about 33 ng/mL, greater than about 34 ng/mL, or greater than about 35 ng/mL) can indicate that the mammal is to be treated with one or more bile acid sequestrants as described herein.

A composition containing a bile acid sequestrant (e.g., colesevelam) can be administered to a mammal in any amount, at any frequency, and for any duration effective to achieve a desired outcome (e.g., to treat diarrhea). In some cases, a composition containing a bile acid sequestrant can be administered to a mammal to reduce colonic transit by 10, 11, 12, 13, 14, 15, 16, 17, 18, 19, 20, 25, 30, 35, 40, 45, 50, 55, 60, 65, 70 percent or more). An effective amount of a composition containing a bile acid sequestrant can be any amount that reduces a mammal's diarrhea without producing significant toxicity to a mammal. Typically, an effective amount of a composition containing a bile acid sequestrant can be any amount greater than or equal to about 250 mg of a bile acid sequestrant (e.g., greater than or equal to about 250, 500, 750, 1000, 1250, 1500, 1750, 2000, or more mg of, for example, colesevelam per administration) provided that that amount does not induce significant toxicity to the mammal upon administration. In some cases, an effective amount of a bile acid sequestrant such as colesevelam can be between 250 mg and 10 g (e.g., between 250 mg and 1250 mg, between 500 mg and 1500 mg, or between 750 mg and 2000 mg). Various factors can influence the actual effective amount used for a particular application. For example, the frequency of administration, duration of treatment, use of multiple treatment agents, route of administration, and severity of the diarrhea may require an increase or decrease in the actual effective amount administered.

The frequency of administration of a composition containing a bile acid sequestrant can be any frequency that reduces a mammal's diarrhea without producing significant toxicity to the mammal. For example, the frequency of administration can be from about three times a day to about twice a week (e.g., once a day). The frequency of administration can remain constant or can be variable during the duration of treatment. For example, a composition containing a bile acid sequestrant can be administered daily, twice a day, five days a week, or three days a week. A composition containing a bile acid sequestrant can be administered for five days, 10 days, three weeks, four weeks, eight weeks, 48 weeks, one year, 18 months, two years, three years, or five years. A course of treatment can include rest periods. For example, a composition containing a bile acid sequestrant can be administered for five days followed by a ten-day rest period, and such a regimen can be repeated multiple times. As with the effective amount, various factors can influence the actual frequency of administration used for a particular application. For example, the effective amount, duration of treatment, use of multiple treatment agents, route of administration, and severity of the diarrhea may require an increase or decrease in administration frequency.

An effective duration for administering a composition containing a bile acid sequestrant can be any duration that reduces a mammal's diarrhea without producing significant toxicity to the mammal. Thus, the effective duration can vary from several days to several weeks, months, or years. In general, the effective duration for the treatment of diarrhea can range in duration from one day to several days to several months. In some cases, an effective duration can be for as long as an individual mammal is alive and suffering from diarrhea. Multiple factors can influence the actual effective duration used for a particular treatment. For example, an effective duration can vary with the frequency of administration, effective amount, use of multiple treatment agents, route of administration, and severity of the diarrhea.

The invention will be further described in the following examples, which do not limit the scope of the invention described in the claims.

### EXAMPLES

### Example 1

### Bile Acids, Colon Transit, and Bile Acid Binding in Unselected Patients with Irritable Bowel Syndrome with Diarrhea

### Study Design, Randomization and Medication

A double-blind, placebo-controlled, parallel-group, randomized study was conducted to evaluate the effects of oral sodium chenodeoxycholate (CDC) or placebo, once daily for 4 days, in healthy volunteers. In a second trial, oral colesevelam hydrochloride or placebo were administered for 12-14 days in patients with diarrhea-predominant irritable bowel syndrome (IBS-D). The study was approved by a review board and all participants signed informed consent. In both studies, allocation was concealed from the study investigators.

In the first trial, participants were randomized to placebo, CDC 500 mg, or 1000 mg (20 per group). Sodium CDC (unconjugated) was purchased from Calbiochem (EMD Biosciences Inc., San Diego, Calif. 92121), and the Mayo pharmacy prepared identical placebo and CDC capsules, all of which were coated with the pH sensitive polymer, methacrylate (Eudragit® S-100, Rohm Pharmaceuticals, Darmstadt D-64293, Germany). The latter dissolves at the neutral pH found in the distal ileum and was used to ensure ileocolonic delivery of the CDC or placebo. All participants were screened for idiopathic bile acid malabsorption (BAM) with fasting serum 7alpha-hydroxy-4-cholesten-3-one (7αC4).

In the second trial, fasting serum 7αC4 and FGF-19 and baseline colonic mucosal permeability were measured in patients with IBS-D, who were then randomized to colesevelam or placebo (1:1). The drug was purchased as Welchol® (Colesevelam HCl) from Daiichi Sankyo Co. Ltd. (Parsippany, N.J.) as 625 mg tablets. The tablets were prepared as 312.5 mg capsules with matching placebo.

The selected doses for CDC and colesevelam were respectively based on the observations of diarrhea demonstrated in the gallstone dissolution trials (Mok et al., Lancet, 304:253-257 (1974)) and on the FDA approved doses in the treatment of hypercholesteremia (Welchol® (Colesevelam HCL)).

An independent statistician, who otherwise was not involved in the study, generated the randomization codes, and the allocation was concealed. All clinical and laboratory study personnel were blinded throughout the study until all data were locked and analyzed. Clinical safety monitoring was conducted by the study investigators throughout the study.

### Participants

All participants had fasting plasma 7α-C4 (C4) measured to assess for underlying bile acid malabsorption and had serum FGF-19 measured. Participants also completed the following questionnaires: Hospital Anxiety and Depression (HAD) Scale (Zigmond and Snaith, Acta Psychiatr. Scand., 67:361-370 (1983)), SCL-90 (somatization) (Derogatis et al., Psychopharmacol. Bull., 9:13-28 (1973)), and bowel function by validated daily diaries including Bristol Stool Form Scale (BSFS) scores (Lewis and Heaton, Scand. J. Gastroenterol., 32:920-924 (1997)).

### Effect of Sodium Chenodeoxycholate in Healthy Subjects

60 patients (mean age 38.7 years, 43 female) were enrolled to the study. Three groups (n=20 each) were randomized to one oral capsule daily containing placebo, 500 mg, or 1000 mg CDC, each for a period of 4 days. Gastrointestinal (GI) and colonic transit was conducted by a scintigraphic method (Burton et al., J. Nucl. Med., 38:1807-1810 (1997) and Cremonini et al., Aliment Pharmacol. Ther., 16:1781-1790 (2002)) during the last 48 hours of drug ingestion.

Effect of Colesevelam Hydrochloride in Patients with IBS-D

24 patients (all female, mean age 42.7 years) were enrolled to the study. Participants with a prior cholecystectomy (total 6 enrolled) were eligible to participate if the history of IBS-D anteceded the surgery and the patients reported no aggravation of IBS symptoms by the surgery. Two groups were randomized, balanced on BMI (<25 or ≧25 kg/m²), to placebo or colesevelam, 1.875 g twice daily, for 12-14 days. Gastrointestinal and colonic transit were assessed by a scintigraphic method (Burton et al., J. Nucl. Med., 38:1807-1810 (1997) and Cremonini et al., Aliment Pharmacol. Ther., 16:1781-1790 (2002)), and colonic permeability were conducted at baseline and during the last 48 hours of the study.

### Daily Stool Diaries

All participants completed bowel function diaries that documented the time of each bowel movement, stool consistency (Bristol Stool Form Scale), the ease of passage (ranging from 1=“manual disimpaction” to 7=“incontinence”), and completeness of evacuation (1=“yes” and 0=“no”). In the first study, patients recorded diaries during at least 3 days of the baseline period and the 4 days of treatment. The diaries were recorded for at least 3 days of the baseline period and during the entire treatment period of the second study.

### Serum 7α-C4 Measurements

The measurement of fasting serum 7α-hydroxy-4-cholesten-3-one (7α-C4) was conducted using high performance liquid chromatography with tandem mass spectrometry (Camilleri et al., Neurogastroenterol. Motil., 2009 Jul. 10), which is a measurement of hepatic cholesterol synthesis and is closely related to the fecal loss of bile acids. This is a valid screening method for identification of BAM (Sauter et al., Dig. Dis. Sci., 44:14-19 (1999)).

### Serum FGF19 Measurements

Serum FGF19 levels were measured by enzyme-linked immunosorbent assay ((ELISA) FGF19 Quantikine ELISA Kit, R&D Systems, Minneapolis, Minn.). Data from 40 healthy controls were used for comparison with patients with IBS-D.

### Gastrointestinal Transit Measurements

An adaptation of an established scintigraphic method was used to measure gastrointestinal and colonic transit (Burton et al., J. Nucl. Med., 38:1807-1810 (1997) and Cremonini et al., Aliment Pharmacol. Ther., 16:1781-1790 (2002)). Patients with IBS-D had baseline measurement of colonic transit since this is known to be a significant covariate in treatment responses.

The primary endpoints were the colonic geometric center (GC) at 24 hours (GC 24) and ascending colon (AC) emptying t_{1/2}. Secondary transit endpoints were colonic GC at 48 hours, gastric emptying t_{1/2}, and colonic filling at 6 hours.

Colonic GC is an important endpoint which has been shown in previous pharmacodynamic studies using the same methods to be responsive to treatment with prokinetics and secretagogues (Camilleri and Talley, Neurogastroenterol. Motil., 16:135-142 (2004)) in patients with constipation-predominant IBS or functional constipation. It was also shown to be responsive to agents that retard colonic transit such as alosetron (Viramontes et al., Am. J. Gastroenterol., 92:2671-2676 (2001)) in IBS-D.

### Colonic Permeability Measurement

In the second trial conducted in patients with IBS-D, colonic permeability was measured at baseline and at the end of the treatment period by the excretion of urine sugars measured by a liquid chromatography/mass spectrometry method (Lostia et al., Clin. Biochem., 41:887-892 (2008)). After oral ingestion of the sugars (mannitol 1 g and lactulose 5 g) in a methacrylate-coated capsule, urine was collected in a 2-hour aliquot from 6 to 8 hours, and a 16-hour collection from 8 to 24 hours after ingestion of the sugars. The total content of each sugar in each aliquot as well as the ratio of the excretion of the two sugars was estimated. The primary permeability endpoint was the 8-24 hour urine mannitol excretion. Secondary endpoints were urine mannitol at 6-8 hours, urine lactulose at 6-8 hours and 8-24 hours, and urine lactulose to mannitol ratio.

### Statistical Analysis and Sample Size Considerations

### Effect of Sodium Chenodeoxycholate

An analysis of covariance (ANCOVA) assessed the treatment effects of CDC dose on the primary endpoints, colonic GC 24 hours and AC emptying t_{1/2}, with age, gender and BMI as covariates. The ANCOVA analysis compared the responses overall among the three (randomly assigned) treatment groups. Specific pairwise comparisons (e.g., each dose of CDC against placebo) were also examined.

Sample sizes selected (Table I) were based on the results of primary endpoints in healthy volunteers previously studied (data show mean±SD). The estimated effect sizes are based on a 2-sample t-test with N=20 per group, where effect size is the difference in group means as a percentage of the corresponding overall mean (shown in Table I). Note that the effect size demonstrable for colonic GC24 hours and for AC t_{1/2} was 34% and 50% respectively. Moreover, the observed variations (COV %) in these two primary endpoints in the subjects randomized to placebo in this study were 39% and 54%, which are very close to the a priori assumed variations (see Table I).

TABLE I

| | | | |
|---|---|---|---|
| | | | Effect size (%) |
| | | COV | demonstrable with |
| Mean | SD | (%) | 80% power, α = 0.05 |

Pooled Data Used to Determine Effect Size Demonstrable
with 20 Healthy Participants per Treatment Group in CDC Study
(data based on prior lab studies in healthy volunteers)

| | | | | |
|---|---|---|---|---|
| | | | | n = 20 per group |
| Ascending colon t ½, hour | 15.4 | 8.5 | 55 | 50 |
| Colon GC 24 hours | 2.05 | 0.77 | 38 | 34 |
| Serum 7α-C4 ng/mL | 17 | 10 | 59 | 71 |

Pooled Data Used to Determine Effect Size Demonstrable
with 12 Participants with IBS-D per Treatment Group in Colesevelam
Study (data based on prior lab studies in patients with IBS-D)

| | | | | |
|---|---|---|---|---|
| | | | | n = 12 per group |
| Ascending colon t ½, hour | 14.9 | 9.2 | 62 | 74 |
| Colon GC 24 hours | 3.53 | 0.87 | 25 | 30 |
| Serum 7α-C4 ng/mL | 17 | 10 | 59 | 71 |
| Urine 8-24 hr Mannitol, | 8.7 | 8.6 | 99 | 115 |
| mg/hour | | | | |
| Urine 8-24 hr Lactulose, | 2.2 | 1.7 | 77 | 91 |
| mg/hour | | | | |

### Effect of Colesevelam Hydrochloride

An ANCOVA was used to compare treatment groups on the primary endpoints of transit (AC t_{1/2} and geometric center at 24 hours) including baseline transit GC24 values and serum 7αC4 as covariates in the analysis (the latter after a rank transformation for skewness). The effects of colesevelam and placebo on urine mannitol excretion and the ratio of lactulose to mannitol excretion over 8-24 hours was also assessed using an ANCOVA with the corresponding baseline values as covariates. In addition, separate ANCOVA models also examined the potential “interaction” of treatment and serum 7αC4 by including a cross-product term (rank transformation of serum 7αC4 values times treatment category, placebo versus colesevelam) in the model for each of the colonic transit endpoints. The test for a significant interaction effect assesses a potential “differential” treatment effect depending on the baseline level of serum 7αC4.

To assess the effects of colesevelam, with 12 subjects per group, there was approximately 80% power to detect a difference between colesevelam and placebo groups of 0.91 in mean colon GC 24 hours values (corresponding to a change of 37%) based on a two-sample t-test at an a level of 0.05 (two-sided) (see Table I).

### Gastrointestinal and Colonic Transit by Scintigraphy

¹¹¹In was adsorbed on to activated charcoal particles and delivered to the colon by means of a methacrylate-coated, delayed-release, oral capsule. The capsule was ingested following an overnight fast. After the capsule emptied from the stomach, a 99 mTc-sulfur colloid radiolabeled meal was ingested. It consisted of two scrambled eggs, one slice of whole wheat bread, and one glass of whole milk. This meal allowed measurement of gastric and small bowel transit. Subjects ingested standardized meals for lunch and dinner at 4 and 8 hours after the radiolabeled meal, respectively. Abdominal scans were obtained every hour for the first 6 hours (the first 4 hours for the assessment of gastric emptying) and at 8, 24, and 48 hours after ingestion of the ¹¹¹In capsule.

### Transit Data Analysis

The counts in the stomach and each of four colonic regions, ascending, transverse, descending, and combined sigmoid and rectum, were quantitated with a variable region of interest program. Counts were corrected for isotope decay, tissue attenuation, and downscatter of ¹¹¹In counts in the 99 mTc window.

Gastric emptying t_{1/2} is a measure of the time for 50% of the radiolabeled meal (identifiable by radiolabeled tracer) to empty from the stomach. Colonic filling at 6 hours, or the proportion of the radiolabeled meal to have reached the colon at 6 hours, is an indirect measurement of small bowel transit time. Overall colonic transit was summarized as the colonic geometric center (GC) at specified times. The GC is the weighted average of counts in the different colonic regions (ascending (AC), transverse (TC), descending (DC), rectosigmoid (RS)) and stool, respectively 1 to 5. At any time, the proportion of counts in each colonic region is multiplied by its weighting factor as follows:

(%AC×1+%TC×2+%DC×3+%RS×4+%stool×5)/100=GC

Thus, a higher GC reflects a faster colonic transit.

Ascending colon emptying was summarized by the t_{1/2} calculated by linear interpolation of values on the AC emptying curve.

### Statistical Power

Table I provides data regarding statistical power for the two trials.

### Results

### Participants, Study Conduct, and Completion

All medical records were screened for major exclusion criteria (i.e., prior gastrointestinal surgery and concomitant medications). Patients' responses to bowel disease questionnaire, HAD scores and SCL-90 also excluded the presence of significant gastrointestinal symptoms in the healthy volunteers or anxiety, depression, poor quality of life or psychopathology that could act as confounders in assessment of the effects of the administered CDC or colesevelam.

### Effect of Sodium Chenodeoxycholate

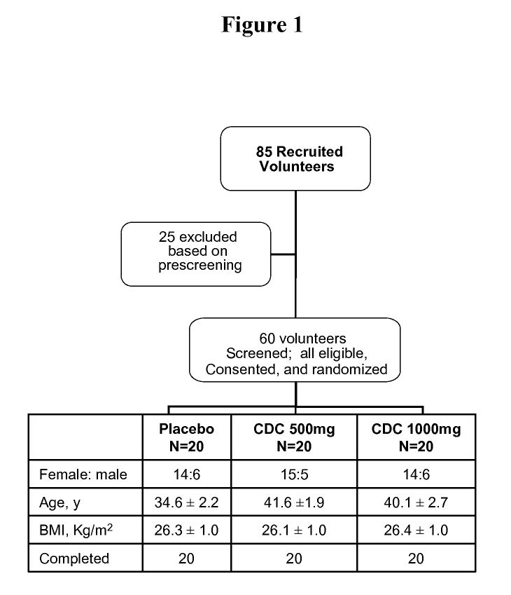

Eighty-five volunteers were recruited for the study through advertisements and mail notifications (FIG. 1). Twenty-five were ineligible based on this initial screen. Of those eligible to participate, all 60 fulfilled the inclusion/exclusion criteria, consented, and were randomized. Demographic data of all participants randomized to CDC or placebo are shown in FIG. 1. They had similar age and BMI.

### Effect of Colesevelam Hydrochloride

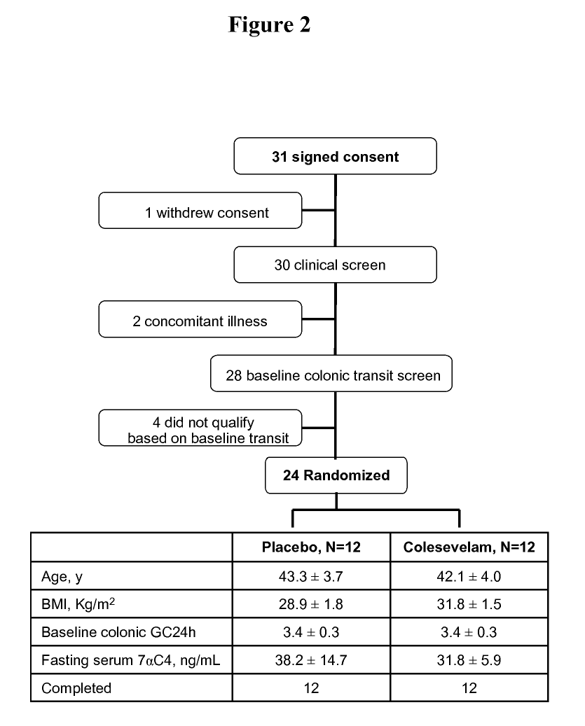

Thirty-one IBS-D patients were recruited and signed consent, and 24 were randomized and completed the study. One withdrew consent prior to the start of the study; two had concomitant illness and were advised not to participate by their primary physicians; and four did not qualify based on baseline transit eligibility criteria (GC 24 hours <2.3) (FIG. 2). Demographic data of all randomized patients are shown in FIG. 2.

All 60 volunteers and 24 IBS-D patients randomized completed the studies, and there was 100% medication compliance (based on coordinator interview and pill count).

Serum 7αC4 and FGF-19 Measurements in Patients with IBS-D and Healthy Controls

A value of <61 ng/mL was established as the 95th percentile for fasting serum 7αC4 level in healthy volunteers. Fifty-five (92%) out of the 60 healthy volunteers and 20 of the 24 IBS-D patients had normal fasting serum 7αC4 values. There were no clinically important differences in the fasting serum 7αC4 levels in the two treatment groups of IBS-D patients (FIG. 2).

The FGF levels in 40 other healthy controls were 161.6 pg/mL (median, IQR 85.9-267.8) and in the 24 patients with IBS-D 105.6 pg/mL (median, IQR 55.8-240.0; p=0.23, Wilcoxon Rank Sum test). Two patients had serum FGF-19 levels below the 5th percentile in health of 36.9 pg/mL.

### Effect of Chenodeoxycholate on Gastrointestinal and Colonic Transit

### Gastric and Small Bowel Transit

Treatment effects of CDC on gastric emptying t_{1/2} and colonic filling at 6 hours were not detected (see Table II).

TABLE II
Effects of NaCDC on Gastrointestinal and Colonic Transit and
Bowel Function (mean ± SEM).

| | Placebo | CDC 500 mg | CDC 1000 mg |
|---|---|---|---|
| | N = 20 | N = 20 | N = 20 |
| GE t_{1/2} (min) | 122.8 ± 6.1 | 126.9 ± 5.3 | 143.0 ± 14.1 |
| CF 6 (%) | 54.6 ± 6.8 | 49.6 ± 7.0 | 46.0 ± 7.1 |
| GC 4 | 0.97 ± 0.24 | 0.87 ± 0.18 | 1.27 ± 0.33 |
| GC 24* | 2.69 ± 0.24 | 2.80 ± 0.27 | 3.76 ± 0.30 |
| GC 48** | 3.76 ± 0.20 | 4.10 ± 0.21 | 4.92 ± 0.05 |
| AC t_{1/2} (hour) | 14.5 ± 1.7 | 12.1 ± 2.1 | 10.7 ± 1.9 |
| Stool frequency per | 1.09 ± 0.13 | 1.50 ± 0.18 | 2.01 ± 0.15 |
| day # | | | |
| Stool consistency by | 3.51 ± 0.16 | 4.29 ± 0.19 | 4.80 ± 0.15 |
| Bristol Stool Form | | | |
| Scale** | | | |
| Ease of passage | 3.9 ± 0.03 | 4.1 ± 0.06 | 4.3 ± 0.06 |
| (scale1-7)** | | | |

*p = 0.01;
**p < 0.0001;
# p < 0.001

### Overall Colonic Transit Time Assessed by Geometric Center

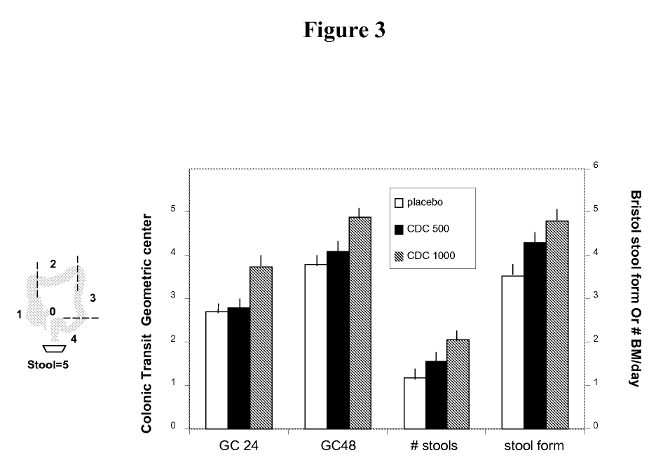

Treatment effects on overall colonic transit were significant at 24 and 48 hours (ANCOVA p=0.01 and <0.0001 respectively), as illustrated in FIG. 3. The effect of the 1000 mg dose was significantly greater than the 500 mg dose on the study primary endpoint, colonic GC at 24 hours (p<0.05).

### Effect of Chenodeoxycholate on Bowel Function in Healthy Volunteers

There were also significant overall treatment effects of CDC (Table II) on stool frequency, consistency, ease of passage (all p<0.001), and on sense of complete evacuation (p=0.02).

### Effect of Colesevelam on Gastrointestinal and Colonic Transit in IBS-D

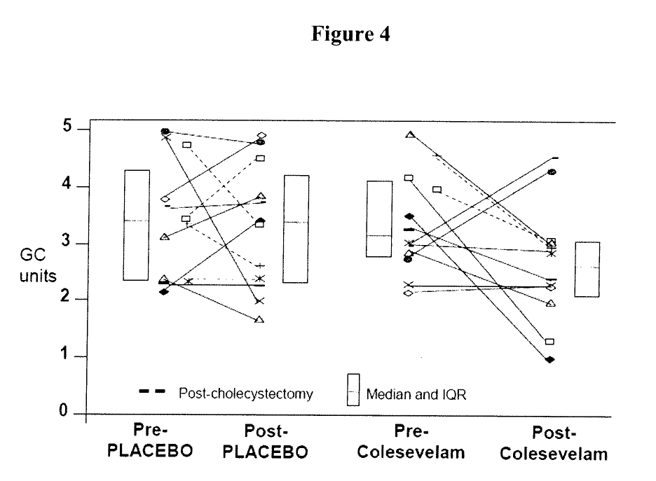

These data are summarized in Table III and FIG. 4.

TABLE III
Effects of Colesevelam on Gastrointestinal and
Colonic Transit (mean ± SEM).

| | Placebo | Colesevelam |
|---|---|---|
| | N = 12 | N = 12 |
| GE t_{1/2} (min)* | 119.6 ± 7.69 | 156.1 ± 17.36 |
| CF 6 (%) | 64.5 ± 8.17 | 58.5 ± 8.72 |
| GC 4 | 0.81 ± 0.19 | 0.42 ± 0.16 |
| GC 24^{$} | 3.30 ± 0.33 | 2.68 ± 0.32 |
| GC 48 | 4.47 ± 0.20 | 4.65 ± 0.13 |
| AC t_{1/2} (hour) | 14.9 ± 3.58 | 18.85 ± 2.88 |
| Stool frequency per day | 2.25 ± 0.34 | 2.14 ± 0.31 |
| Stool consistency by Bristol | 4.57 ± 0.35 | 3.78 ± 0.27 |
| Stool Form Scale*** | | |
| Ease of passage (scale1-7)** | 4.39 ± 0.11 | 4.18 ± 0.14 |

*p = 0.14;
^{$}p = 0.18
**p = 0.047;
***p = 0.12

### Gastric, Small Bowel and Overall Colonic Transit

Colesevelam moderately delayed gastric emptying (proportion emptied at 4 hours: p=0.06; GE t_{1/2} p=0.14) and did not have a significant effect on small bowel transit. There was a tendency for colesevelam to slow colonic transit at 24 hours compared with placebo treatment (ANCOVA using baseline GC24, BMI and serum 7αC4 as covariates; p=0.22). Seven of 12 in the colesevelam group and four of 12 in the placebo group demonstrated a decrease of GC24 of at least 0.7 GC units. Appreciable retardation of colonic transit was observed at 6 and 8 hours (data not shown).

There were four patients in the placebo group and two in the colesevelam group who had prior cholecystectomy. Two of the four patients with prior cholecystectomy in the placebo group and the two in the colesevelam group had >0.7 GC unit slowing of colonic transit at 24 hours. Thus, cholecystectomy state did not appear to have a significant effect on the results (FIG. 4).

### Ascending Colonic Emptying

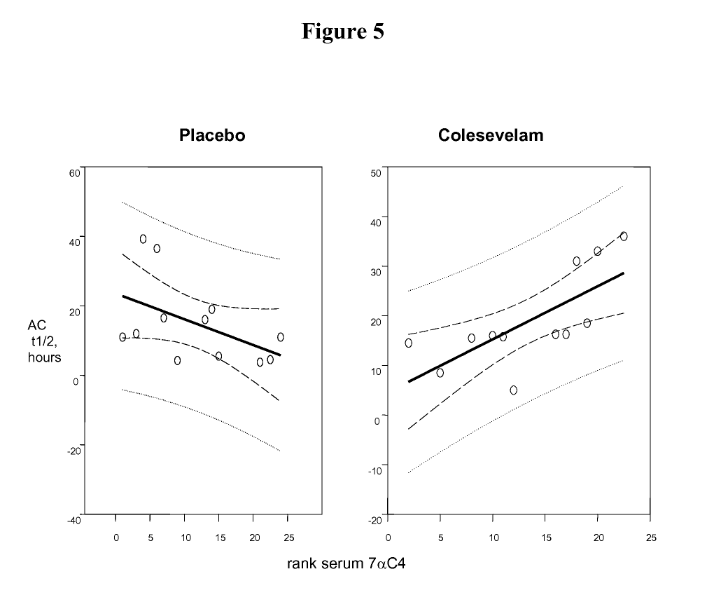

On average, AC t_{1/2} was >4 hours slower in the colesevelam group compared to the placebo group. There was a statistically significant interaction between treatment and baseline serum 7αC4 (p=0.0025). FIG. 5 shows the relationship between (the rank transformation of) baseline serum 7αC4 and observed AC t_{1/2} values, separately for the two treatment groups. In the colesevelam group, the Spearman correlation coefficient was 0.87 (p<0.001), and in the placebo group the coefficient was −0.49 (p=0.11). Thus, the higher the baseline serum 7αC4, the higher was the observed AC t_{1/2} after colesevelam treatment.

### Effect of Colesevelam on Bowel Function and Colonic Mucosal Permeability in IBS-D

Colesevelam had no effect on the number of bowel movements per day, but there was a tendency to improve stool consistency (p=0.12) and ease stool passage (p=0.048).

Urinary excretion of mannitol 8-24 hours after ingestion was not significantly associated with fasting serum 7αC4 (Spearman rank correlation −0.18, p=0.41). There were no clinically important differences in permeability measurements in the two groups at baseline (data not shown). There was also no significant treatment effect on colonic mucosal permeability (Table IV).

TABLE IV
Post-treatment Measurements of Colonic Permeability (mean ± SEM).

| | Placebo | Colesevelam | P |
|---|---|---|---|
| Urinary excretion of mannitol | 45.8 ± 8.8 | 64.3 ± 13.3 | 0.28 |
| 8-24 hours, mg | | | |
| Urinary excretion of lactulose | 19.9 ± 3.12 | 28.5 ± 5.9 | 0.30 |
| 8-24 hours, mg | | | |
| L:M Ratio (8-24 hours) | 0.49 ± 0.05 | 0.59 ± 0.19 | 0.62 |

Adverse Events with Sodium Chenodeoxycholate and Colesevelam

The most common adverse events for CDC were loose stools, diarrhea and lower abdominal cramps, each being more frequently observed in the CDC 1000 mg group (15-75%). Headache was observed at similar rates (20-25%) between the placebo and two treatment groups. Gas, bloating and nausea were experienced by 5-20% of the participants.

In the colesevelam study, the most common adverse events were headache, nausea, flatulence and green colored stools, which each occurred at similar rates in the placebo and treatment groups (10-45%). Smaller numbers experienced uterine and abdominal cramps and urinary tract infection. There were no serious adverse events, and no participant had to stop the study due to an adverse event in either of the two studies.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a diarrhea condition, said method comprising administering to a mammal having said diarrhea condition a composition comprising a bile acid sequestrant.

2. The method of claim 1, wherein said mammal is a human.

3. The method of claim 1, wherein said diarrhea condition is bile acid malabsorption induced diarrhea.

4. The method of claim 1, wherein said bile acid sequestrant is colesevelam.

5. The method of claim 1, wherein said composition is administered to said mammal under conditions wherein the amount of said bile acid sequestrant in said composition is between about 1 gram and about 5 grams.

6. The method of claim 5, wherein said composition is administered to said mammal between once every other day to four times a day.

7. The method of claim 1, wherein said method comprises assessing said mammal before said administering step to determine if said mammal comprises a serum level of 7αC4 greater than about 25 ng/mL of serum, wherein the presence of said serum level indicates that said mammal is to be administered said bile acid sequestrant.

8. The method of claim 7, wherein said serum level of 7αC4 is a fasting serum level of 7αC4.

9. The method of claim 1, wherein said method comprises assessing said mammal before said administering step to determine if said mammal comprises a serum level of 7αC4 greater than about 30 ng/mL of serum, wherein the presence of said serum level indicates that said mammal is to be administered said bile acid sequestrant.

10. A method for treating a diarrhea condition, said method comprising (a) identifying a mammal having said diarrhea condition and a serum level of 7αC4 greater than about 25 ng/mL of serum, and (b) administering a composition comprising a bile acid sequestrant to said identified mammal under conditions wherein the severity of said diarrhea condition is reduced.

11. The method of claim 10, wherein said serum level of 7αC4 is a fasting serum level of 7αC4.

12. A method for treating a diarrhea condition, said method comprising (a) identifying a mammal having (i) said diarrhea condition, (ii) a serum level of 7αC4 greater than about 20 ng/mL of serum, and (iii) a potential bile acid malabsorption condition based on a fecal bile acid excretion test or ⁷⁵SeHCAT retention test, and (b) administering a composition comprising a bile acid sequestrant to said identified mammal under conditions wherein the severity of said diarrhea condition is reduced.

13. The method of claim 12, wherein said fecal bile acid excretion test is a 48 hour fecal bile acid excretion test.

14. The method of claim 12, wherein said ⁷⁵SeHCAT retention test is a ⁷⁵SeHCAT retention test at 7 days.
